(19) United States
(12) Patent Application Publication
(10) Pub. No.: US 2023/0073317 A1
(43) Pub. Date: Mar. 9, 2023

(54) MINERALOGICAL TEST SAMPLING AND ANALYSIS

(71) Applicant: SGS North America Inc., Rutherford, NJ (US)

(72) Inventor: Tomas Hrstka, Prague (CZ)

(21) Appl. No.: 17/583,686

(22) Filed: Jan. 25, 2022

Related U.S. Application Data
(60) Provisional application No. 63/141,201, filed on Jan. 25, 2021.

(51) Int. Cl. G01N 33/24

(52) U.S. Cl. CPC G01N 33/24

(57) ABSTRACT

Disclosed is a test unit for automated mineralogical spectral analysis of a designated industrial value-containing material, comprising a particle-attractant substrate with a monolayer of sized sample particles of the designated industrial value-containing material thereon, wherein the monolayer is configured to present the sized sample particles with an outer surface profile which is visible for the spectral analysis.

### REFERENCE TO CO-PENDING APPLICATION

This application claims the benefit of priority under 35 U.S.C. § 119(e) to U.S. Provisional Patent Application No. 63/141,201, filed Jan. 25, 2021 and entitled MINERALOGICAL TEST SAMPLING AND ANALYSIS, and the entire disclosure set forth therein is incorporated herein by reference.

### FIELD OF THE DISCLOSURE

The present disclosure relates to mineralogical spectral analysis.

### BACKGROUND

In commodity-driven businesses, such as those in mining industries involving the processing of mineral and material particles, profitability is directly tied to the balance between minimizing costs of production while optimizing the performance of a finished product. Thus, considerable value is placed in the analytical techniques used to determine mineral/material compositions, and structural characteristics of the particles, to predict their behavior during processing and/or refining. Classical approaches for automated mineralogy (“AM”) to study particulate material use a block of randomly distributed sample particles mixed in epoxide resin. Each such epoxide resin block is then ground and polished to produce a single two-dimensional (“2D”) crosscut sample surface which thus cuts through the random distribution of particles. Each such crosscut sample surface is then presented to an analytical technique such as by way of an automated scanning electron microscope (“A-SEM”) for collection of mineral abundance (phase proportions, indicative of a quantity of individual phases present), and composition data. Combinations of SEM Backscattered electrons (“BSE”) and Energy-dispersive X-ray spectroscopy (“EDS”) data are generally used in this regard.

Conventional approaches to prepare the 2D polished crosscut sample surfaces, as briefly outlined above, tend to be relatively time-intensive, costly and/or cumbersome. Often, a considerable period of time is required to prepare, mix, cure, grind and polish the crosscut sample surfaces, which can become an operational bottleneck particularly in operations requiring high volume (or throughput) of representative crosscut samples to be produced for AM analysis.

The exposure of the particle sections (in the crosscut sample surface) to grinding and polishing media can also degrade the crosscut samples themselves, causing them to potentially yield inaccurate or unrepresentative data. For example, water-soluble mineral constituents in an exposed particle section may be inadvertently dissolved in preparing the crosscut sample surface, or granular sub-particle constituents physically integral to the sample particles can be inadvertently dislodged from the exposed particle sections in the crosscut sample surface, during the physical actions associated with cutting and polishing the crosscut sample surface.

Further, conventional techniques are generally based on the objective of obtaining a substantially flat crosscut sample surface, particularly for EDS analysis, where the flatter and more continuous the crosscut surface, the higher the accuracy of the results on the properties and chemical analysis of the exposed particle section. Meanwhile, such analyses, particularly for textural and complexity (shape & complexity) data, remain impractical, or at best of limited value, due to the stereological constraints of a two dimensional crosscut sample in an attempt to represent characteristics of 3D particles.

It may thus be desirable to provide novel approaches for AM analysis and/or for preparing samples therefor, or at least to provide the public with one or more useful alternatives.

### SUMMARY

An aspect may provide a test unit for automated mineralogical spectral analysis of a designated industrial value-containing material, the test unit comprising a particle-attractant substrate with a monolayer thereon, the monolayer comprising sized sample particles of the designated industrial value-containing material, wherein the monolayer is configured to present the sized sample particles with an outer surface profile which is visible for the spectral analysis.

In some example embodiments, the sized sample particles may be dispersed on the particle-attractant substrate.

In some example embodiments, the monolayer may include dispersant particles interspersed with the sized sample particles.

In some example embodiments, the sized sample particles may form a sized subset of sample particles from a collection of sample particles of the designated industrial value-containing material.

In some example embodiments, the sized subset of sample particles may have a designated size distribution based on a designated size ranging from about 1 to about 2400 μm in maximum diameter.

In some example embodiments, the sized subset of particles may have a designated size ranging from any one of:
from about 1 μm to about 38 μm;
from about 38 μm to about 75 μm;
from about 75 μm to about 150 μm;
from about 150 μm to about 300 μm;
from about 300 μm to about 600 μm;
from about 600 μm to about 1200 μm, and
from about 1200 μm to 2400 μm.

In some example embodiments, the sized subset of particles may have a designated size distribution according to the formula:

SSPS_{MAX}=(SSPS_{MIN})A

Where
SSPS_{MAX} is the sized sample particle maximum size;
SSPS_{MIN} is the sized sample particle minimum size; and
1.0<A<10.0.

In some example embodiments, the dispersant particles may have a designated size ranging from about 10 percent to about 90 percent of a designated size of the sized sample particles.

In some example embodiments, the dispersant particles may have a designated size distribution of about 30 percent to about 90 percent of the corresponding sized sample particles.

In some example embodiments, the dispersant particles may have a designated size distribution according to the formula:

DPS_{MAX}=(SSPS_{MAX})B

Where
DPS_{MAX} is the dispersant particle maximum size;
SSPS_{MAX} is the sized sample particle maximum size; and
0.1<B<0.9.

In some example embodiments, the dispersant particles may be selected from one or more of graphite, salt, epoxy resin, glass, ceramic, metallic, organic, inorganic and/or polymeric materials.

In some example embodiments, the sized sample particles may originate from one or more steps of physical separation based on a dimensional value of the sample particles.

In some example embodiments, the one or more steps of physical separation may include one or more of wet screening, dry screening and/or cyclosizing.

In some example embodiments, the sized sample particles may be fixed to the particle-attractant substrate.

In some example embodiments, the particle-attractant substrate may include an adhesive layer, with the sized sample particles adhered thereto.

In some example embodiments, the particle-attractant substrate may include a magnetically or statically charged surface, with the sized sample particles adhered thereto.

In some example embodiments, the sized sample particles and dispersant particles may be fixed to the particle-attractant substrate.

In some example embodiments, the particle-attractant substrate may include an adhesive layer, with the sized sample particles and dispersant particles adhered thereto.

In some example embodiments, the particle-attractant substrate may include a magnetically or statically charged surface, with the sized sample particles and dispersant particles adhered thereto.

In some example embodiments, the particle-attractant substrate may include a plurality of dispersed sites, each to receive a corresponding sized sample particle.

In some example embodiments, the dispersed sites may be defined by localized particle receiving sectors.

In some example embodiments, the particle receiving sectors may be provided by a plurality of adhesive dots.

In some example embodiments, the particle receiving sectors may be provided by a mesh structure applied to the substrate.

Another aspect may provide a method of preparing a test unit for mineralogical spectral analysis of a designated industrial value-containing material, comprising depositing sized sample particles of the designated industrial value-containing material on a particle-receiving surface defined on a particle-attractant substrate to form a monolayer thereon, the monolayer comprising the sized ample particles, wherein the monolayer is configured to present the sized sample particles with respective outer surface profiles which are visible for the spectral analysis.

In some example embodiments, the depositing may include dispersing the sized sample particles on the particle-attractant surface.

Some example embodiments may further comprise interspersing dispersant particles with the sized sample particles in the monolayer.

Some example embodiments may further comprise forming the sized sample particles as a sized subset of sample particles from a collection of sample particles of the designated industrial value-containing material.

In some example embodiments, the interspersing may include pre-mixing the dispersant particles with the sized sample particles.

In some example embodiments, the sized subset of sample particles may have a size distribution based on a designated size ranging from about 1 to about 2400 μm in maximum diameter.

In some example embodiments, the sized subset of particles may have a designated size ranging from any one of:
from about 1 μm to about 38 μm;
from about 38 μm to about 75 μm;
from about 75 μm to about 150 μm;
from about 150 μm to about 300 μm;
from about 300 μm to about 600 μm;
from about 600 μm to about 1200 μm, and
from about 1200 μm to 2400 μm.

In some example embodiments, the sized subset of particles may have a designated size distribution according to the formula:

SSPS_{MAX}=(SSPS_{MIN})A

Where
SSPS_{MAX} is the sized sample particle maximum size;
SSPS_{MIN} is the sized sample particle minimum size; and
1.0<A<10.0.

In some example embodiments, the dispersant particles may have a designated size ranging from about 10 percent to about 90 percent of the designated size of the sized sample particles.

In some example embodiments, the sized sample particles and/or dispersant particles may have a designated size distribution of about 30 percent to about 90 percent.

In some example embodiments, the dispersant particles may have a designated size distribution according to the formula:

DPS_{MAX}=(SSPS_{MAX})B

Where
DPS_{MAX} is the dispersant particle maximum size;
SSPS_{MAX} is the sized sample particle maximum size; and
0.1<B<0.9.

In some example embodiments, the dispersant particles may be selected from one or more of graphite, hardened milled epoxy resin, hardened glass, ceramic, metallic, organic, inorganic and/or polymeric materials.

Some example embodiments may further comprise fixing the sized sample particles to the particle-attractant substrate.

In some example embodiments, the particle-attractant substrate may include an adhesive layer, wherein the fixing includes adhering the sized sample particles thereto.

Some example embodiments may further comprise fixing the sized sample particles and dispersant particles to the particle-attractant substrate.

In some example embodiments, the particle-attractant substrate may include an adhesive layer, wherein the fixing includes adhering the sized sample particles and dispersant particles thereto.

Some example embodiments may further comprise wet screening, dry screening and/or cyclosizing the material to obtain the sized sample particles.

In some example embodiments, the interspersing may include depositing dispersant particles with the sample particles on the particle-receiving surface.

Some example embodiments, prior to the depositing of the dispersant particles, may further comprise mixing the sample particles with the dispersant particles.

Some example embodiments may further comprise forming a plurality of dispersed sites on the particle-attractant substrate, for each to receive a corresponding sized sample particle.

In some example embodiments, the forming may include forming localized particle receiving sectors.

In some example embodiments, the forming may include forming a plurality of adhesive dots.

In some example embodiments, the forming may include applying a mesh structure to the substrate.

Another aspect may provide a test unit for mineralogical spectral analysis of a designated industrial value-containing material, the test unit comprising a particle-attractant substrate with a monolayer thereon, the monolayer comprising sized sample particles of the designated industrial value-containing material dispersed, wherein at least some of the sample particles in the monolayer are dispersed and the monolayer is configured to present the sized sample particles with an outer surface profile which is visible, within a depth of field range of one or more detectors, or otherwise suitable for the spectral analysis.

Another aspect may provide a test unit for mineralogical spectral analysis of a designated industrial value-containing material, comprising a particle-attractant substrate with a monolayer, the monolayer comprising sized sample particles of the designated industrial value-containing material thereon, wherein the monolayer is configured to present the sized sample particles with respective outer surface profiles which are visible, within a designated depth of field range of one or more spectral analysis detectors.

In some example embodiments, the depth of field range may include an outer limit beyond an outer extremity of the particles, and an inner limit intermediate the outer extremity and an inner extremity.

In some example embodiments, the inner extremity may correspond to a reference plane or surface of the particle-attractant substrate.

Another aspect may provide a method for analyzing an industrial value-containing material, comprising initiating spectral analysis on a test unit as defined in any one or more of the claims, clauses, examples, or elsewhere in the present disclosure.

Another aspect may provide a method for analyzing an industrial value-containing material, comprising preparing a test array of one or more test units as defined in any one or more of the claims, clauses, examples, or elsewhere in the present disclosure, wherein each of the one or more test units in the test array includes a plurality of corresponding sized sample particles of a designated size range, initiating spectral analysis on the test array to collect data on each of the at least one test unit, and determining value-containing material content and/or properties therefrom.

Some example embodiments may further comprise assembling datasets for each test unit, so as to create a number of datasets for the test array, and following the initiating, forming a cumulative dataset for the test array from the number of datasets.

Some example embodiments may further comprise configuring one or more spectral analysis detectors with a designated depth of field range to collect data from a visible outer surface of the sized particles.

In some example embodiments, the configuring may include setting a depth of field outer limit to be beyond an outer extremity of the particles, and a depth of field inner limit intermediate the outer extremity and an inner extremity.

In some example embodiments, the configuring may include setting the depth of field inner limit to correspond to a reference plane or of the particle-receiving substrate.

Another aspect may provide a test unit for automated mineralogical spectral analysis of a designated industrial value-containing material, comprising a particle-attractant substrate with a monolayer of sized sample particles of the designated industrial value-containing material thereon, wherein the monolayer is configured to present the sized sample particles with an outer surface profile which is visible for the spectral analysis.

Another aspect may provide a test unit for automated mineralogical spectral analysis of a designated industrial value-containing material, comprising a particle-attractant substrate with a monolayer segment of sized sample particles of the designated industrial value-containing material thereon, wherein the monolayer segment is configured to present the sized sample particles with an outer surface profile which is visible for the spectral analysis.

In some example embodiments, the least one monolayer segment may include a first monolayer segment of sized sample particles of a first size range and a second monolayer segment of sized sample particles of a second size range.

Some example embodiments may further comprise a plurality of dispersant particles interspersed with the sized sample particles in the monolayer.

In some example embodiments, the dispersant particles may be transient and selected from any one or more of particles that are soluble, volatile, removable, or configured to vaporize or disintegrate.

In some example embodiments, the dispersant particles are formed from salt or dry ice.

In some example embodiments, the designated industrial value-containing material may comprise one or more of:
non-metallic ores including limestone, manganese, mica, gypsum, coal, dolomite, phosphate, salt, granite;
metal ores including iron, nickel, lead, zinc copper, aluminium, tin, tungsten, molybdenum, tantalum, cobalt, bismuth, cadmium, titanium, zirconium, antimony, manganese, magnesium, lithium, uranium, thorium, beryllium, chromium, germanium, vanadium, gallium, hafnium, indium, niobium, rhenium, and thallium;
precious metal ores including gold, silver, palladium, and the platinum group metals including ruthenium, rhodium, palladium, osmium, iridium, and platinum,
precious stones including diamond, ruby and/or sapphire;
host materials of one or more metal, precious metals, precious stones, in one or more feed, concentrate and/or tailings forms; and
processing, smelting, rafting products/materials thereof including, without limitation, matte, slag and/or slimes.

Another aspect may provide a method of preparing a test unit for mineralogical spectral analysis of a designated industrial value-containing material, comprising depositing sized sample particles of the designated industrial value-containing material on a particle-receiving surface defined on a particle-attractant substrate to form a monolayer of the sized sample particles thereon, wherein the monolayer is configured to present the sized sample particles with respective outer surface profiles which are visible for the spectral analysis.

Another aspect may provide a test unit for automated mineralogical spectral analysis of a designated industrial value-containing material, comprising a particle-attractant substrate with a monolayer, at least in part, of sized sample particles of the designated industrial value-containing material thereon, wherein the monolayer is configured to present the sized sample particles with an outer surface profile which is visible for the spectral analysis, which may provide a partial three dimensional view (or alternatively expressed as a 2.5 dimensional view) of a profile of the sized sample particles which are visible for the analysis, which may encompass the surface region of the sample particle that is oriented so as to be exposed to a visible field of propagation of incident electron beam radiation directly or indirectly from an electron beam emitter for the analysis, so as to reflect (or generate or scatter) a signal collectable by one or more detectors, which may not, in some cases, include the outer surface profile of a sample particle that is in the shadow of another sample particle and which thus may not receive incident electron radiation from an electron beam, and wherein an extent of the profile of the sample may depend on the number of electron beam sources or detector configurations deployed in the analysis.

Another aspect may provide a test unit for automated mineralogical spectral analysis of a designated industrial value-containing material, comprising a particle-attractant substrate with a monolayer, at least in part, of sized sample particles of the designated industrial value-containing material thereon, wherein the monolayer is configured to present the sized sample particles with an outer surface profile which is visible for the spectral analysis, wherein the monolayer provides a collection or array of the sized sample particles in which substantially all of the sample particles lie on a common surface, and/or along a common plane and/or along a common axis defined thereon.

Another aspect may provide a test unit for automated mineralogical spectral analysis of a designated industrial value-containing material, comprising a particle-attractant substrate or surface with a monolayer, at least in part, of sized sample particles of the designated industrial value-containing material thereon, wherein the monolayer is configured to present the sized sample particles with an outer surface profile which is visible for the spectral analysis, wherein the particle-attractant substrate or surface is configured to retain, temporarily or otherwise, the sized sample particles in position on the subject surface, for example by way of adhesion, static attraction and/or magnetic attraction.

Another aspect may provide a test unit for automated mineralogical spectral analysis of a designated industrial value-containing material, comprising a particle-attractant substrate or surface with a monolayer, at least in part, of sized sample particles of the designated industrial value-containing material thereon, wherein the monolayer is configured to present the sized sample particles with an outer surface profile which is visible for the spectral analysis, wherein the sized sample particles are configured to be a specified size, shape or orientation, or other characteristic within one or more designated dimensional or other parameters, such as diameter, thickness, and/or length, that may form a substantially homogenous grouping of sample particles or a substantially heterogeneous grouping of sample particles and/or dispersant particles.

Another aspect may provide a test unit for automated mineralogical spectral analysis of a designated industrial value-containing material, comprising a particle-attractant substrate or surface with a monolayer, at least in part, of sized sample particles of the designated industrial value-containing material thereon, wherein the monolayer is configured to present the sized sample particles with an outer surface profile which is visible for the spectral analysis, wherein the sized sample particles are configured according to a maximum size, a minimum size or an average or median size, such as diameter, thickness and/or length, which may be defined by a sizing, separation, selection and/or isolation device or technique, such as a sieve or screen and may be expressed in units associated with a size dimension of the particles or of a sizing, separation and/or isolation device, for example by the measurement of openings in a sieve or mesh screen.

Another aspect may provide a test unit for automated mineralogical spectral analysis of a designated industrial value-containing material, comprising a particle-attractant substrate or surface with a monolayer, at least in part, of sized sample particles of the designated industrial value-containing material thereon, wherein the monolayer is configured to present the sized sample particles with an outer surface profile which is visible for the spectral analysis, wherein the sized sample particles may be configured to be accessible or exposed so as to receive and reflect (or scatter) incident radiation received directly or indirectly from an electron beam or other radiation in the analysis.

### BRIEF DESCRIPTION OF THE FIGURES

Several example embodiments of the present disclosure will be provided, by way of examples only, with reference to the appended drawings, wherein:

FIG. 1 is a schematic view of a scheme to produce several test units;

FIG. 2 is a schematic view of several test environments;

FIGS. 3a, 3b and 4 are schematic views of several examples of test units;

FIGS. 5 to 7 are schematic views of schemes to prepare the test units of FIG. 1;

FIG. 8 is a schematic view of a spectral analysis test environment;

FIG. 9 is a perspective view of a spectral analysis installation; and

FIGS. 10a, 10b and 11 are images of results from spectral analyses on several test units.

### DETAILED DESCRIPTION

It should be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical, mechanical or other connections or couplings. The terms upper, lower, and vertical are intended for operative context only and are not necessarily intended to limit the invention only to those configurations or orientations. In any instance in which the disclosure refers to a single instance of an element, example embodiments may include a multiple of such elements. The term “or” is not necessarily intended to mean in the exclusive alternative, but may also be in the inclusive alternative so as to be the equivalent of “and/or”. The term “at least one” in reference to any element is not intended to force an interpretation on any other reference elsewhere in the disclosure to a single instance of an element to mean only one such instance of the element. Furthermore, and as described in subsequent paragraphs, the specific mechanical and/or other configurations illustrated in the drawings are intended to exemplify embodiments of the invention. However, other alternative mechanical and/or other configurations are possible which are considered to be within the teachings of the instant disclosure. Furthermore, the present disclosure provides basis for any one element, feature, structure, function, of any aspect and/or example embodiment described in the present disclosure including the figures, clauses and/or claims herein, to be claimed on its own or be combined with any one or more elements, features, structures, functions, and/or steps from the same or any other aspects and/or example embodiment described in the present disclosure including the figures, clauses and/or claims herein.

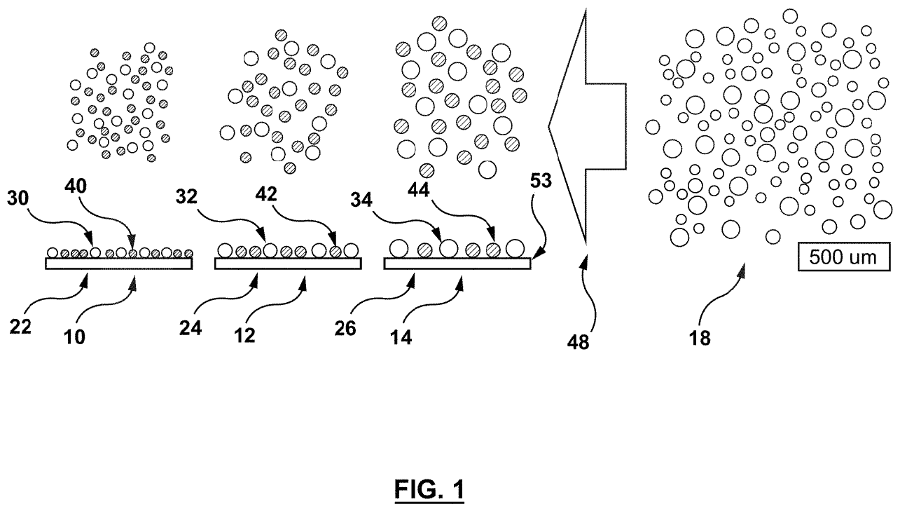

FIG. 1 illustrates an example embodiment in which several test units, in this case first, second and third test units 10, 12, 14, are prepared for AMS analysis of a collection of sized sample particles of a designated industrial value-containing material as shown at 18. Each test unit 10, 12, 14 provides a respective particle-attractant substrate 22, 24, 26, each with corresponding first, second and third monolayers 30, 32, 34 of correspondingly sized sample particles of the designated industrial value-containing material 18 dispersed thereon. For example, the monolayer is be configured to present the sized sample particles with an outer surface profile which is visible for the spectral analysis, wherein the monolayer provides a collection or array of the sized sample particles in which substantially all of the sample particles lie on a common surface, and/or along a common plane and/or along a common axis defined thereon. The particle-attractant substrate or surface may be configured to retain, temporarily or otherwise, the sized sample particles in position on the subject surface, for example by way of adhesion, static attraction and/or magnetic attraction.

As will be described, for example, the sized sample particles are configured according to a maximum size, a minimum size or an average or median size, such as diameter, thickness and/or length, which may be defined by a sizing, separation, selection and/or isolation device or technique, such as a sieve or screen and may be expressed in units associated with a size (or other physical property) dimension of the particles or of a sizing, separation and/or isolation device, for example by the measurement of openings in a sieve or mesh screen. Each monolayer 30, 32, 34 may be configured to present the correspondingly sized sample particles with an outer surface profile visible for AMS analysis. For example, the sized sample particles may be configured to be accessible or exposed so as to receive and reflect (or scatter) incident radiation received directly or indirectly from an electron beam or other radiation in the analysis. For illustration purposes, the sample particles are shown schematically as being spherical, and having the same size, though in actual fact they may tend to have randomly different shape configurations, and thus may each be randomly oriented in the monolayer, and may have, in most cases, an overall size falling inside a designated size range, in this case three different and progressively larger size ranges from the first monolayer 22 to the third monolayer 26. In this example embodiment, the test units 10, 12, 14 provide the basis for three separate analyses of the value-containing material at different size ranges, wherein each respective size range may yield unique data concerning the value-containing material. Such data may then be analyzed separately, or re-combined for the purposes of further analysis to provide data for the original unsized sample 18.

Examples of the suitable industrial value-containing materials which may be used in mineralogical spectral analysis may include, but not be limited to: i) non-metallic ores (limestone, manganese, mica, gypsum, coal, dolomite, phosphate, salt, granite), ii) metal ores (iron, nickel, lead, zinc copper, aluminium, tin, tungsten, molybdenum, tantalum, cobalt, bismuth, cadmium, titanium, zirconium, antimony, manganese, magnesium, lithium, uranium, thorium, beryllium, chromium, germanium, vanadium, gallium, hafnium, indium, niobium, rhenium, and thallium), iii) precious metal ores (Gold, silver, palladium, and the platinum group metals including ruthenium, rhodium, palladium, osmium, iridium, and platinum); iv) materials containing precious stones such as diamond, ruby, sapphire; their related host rocks and processed related equivalents like feed, concentrate and tailings; and any of their processing, smelting, rafting products thereof (e.g. matte, slag, slimes etc.) In general, those which can be broken, milled, ground, or otherwise prepared to more or less equant shapes (having diameters approximately equal, so as to be roughly cubic or spherical) may, in some instances, be more preferred candidates. Meanwhile, those materials with highly elongated shapes (such as those which are needle like or flakey before or after preparation) may be less preferred in some instances.

The particle-attractant substrate 22, 24, 26 may be provided in a number of forms, including, without limitation, single-sided or double-sided tape, such as those currently used in SEM and EDS applications, for example those commercially available from https://www.agarscientific.com/carbon-conductve-adhesive-tapes, and those commercially available under the brand PELCO Tabs™. Such substrates may also include, or alternatively be placed on, a sample carrying media such as glass, metal, polymer to provide flat, curved, angular or other configurations which can support or receive one or more of the monolayers 30, 32, 34. Lacey carbon support films can be also used as a particle-attractant substrate for superfine particles. Further, such substrates may be provided with one or more layers of one or more coatings including instances where the monolayer may penetrate at least one of the layers, in a manner to secure the particle either permanently or temporarily to the substrate, while enabling the particles to present their respective exposed surfaces (that is their respective outer surface profiles) in their original or native form following the formation of the sample particles 18 and after the sizing step.

In some example embodiments, each of the first, second and third monolayers 30, 32, 34 may be prepared by subjecting the sample particles 18 to one or more steps of physical separation (collectively shown schematically by arrow 48), which may be based a size and/or shape, or other dimensional parameter of the sample particles, as schematically represented by arrow 48. The one or more steps of physical separation may include, for example, one or more of wet screening, antistatic (or other forms of) dry screening and/or cyclosizing to form a sized subset of particles for each monolayer 30, 32, 34, of a respective collection of sample particles of the designated industrial value-containing material, as defined by one or more of size and/or a shape, or other dimensional parameter of the sample particles therein. As a non-limiting example, wet screening may include adding water (or other liquids) to a screen to increase its capacity and/or improve its sizing efficiency. Water (or other liquids) may be introduced either by adding it to a feedstock or by spraying it over a sample material on the screen, and/or by another mode. As another non-limiting example, anti-static dry screening may include using sieves in an anti-static environment, for example as provided by a use of screens which are coated with antistatic materials such as conductive plastic or metal to minimize, if not prevent, static attachment of relatively smaller particles on the surface of relatively larger particles or agglomeration of relatively smaller particles to form conglomerates due to static attachment. As another nonlimiting example, cyclosizing may in some cases include using of centrifugal force to separate particles based on their size and/or densities and/or shape, for example by the use of a hydraulic cyclone elutriator as described by Kelsall and McAdam (1963) Transactions of the Institution of Chemical Engineers, Volume: 41, Pages: 84-95.

As shown in FIG. 1, each of the first, second and third monolayers 30, 32, 34 may include first, second and third distributions of dispersant particles shown at 40, 42 and 44 respectively interspersed with the sized sample particles. For illustration purposes, the dispersant particles 40, 42, 44 are shown schematically as being spherical and having the same but smaller size, when compared to the sized sample particles in the corresponding monolayer. In some example embodiments, the dispersant particles 40, 42, 44 may also have different shape configurations and may have an overall size falling inside a designated size range, which is smaller than the designated size range of the sized sample particles (such as, that the individual sizes of the dispersant particles may fall inside a range of sizes that are smaller than those in the range of sizes of the sized sample particles). The dispersant particles may also have three different and progressively larger size (or other dimensional parameter) ranges from the first monolayer 30 to the third monolayer 34.

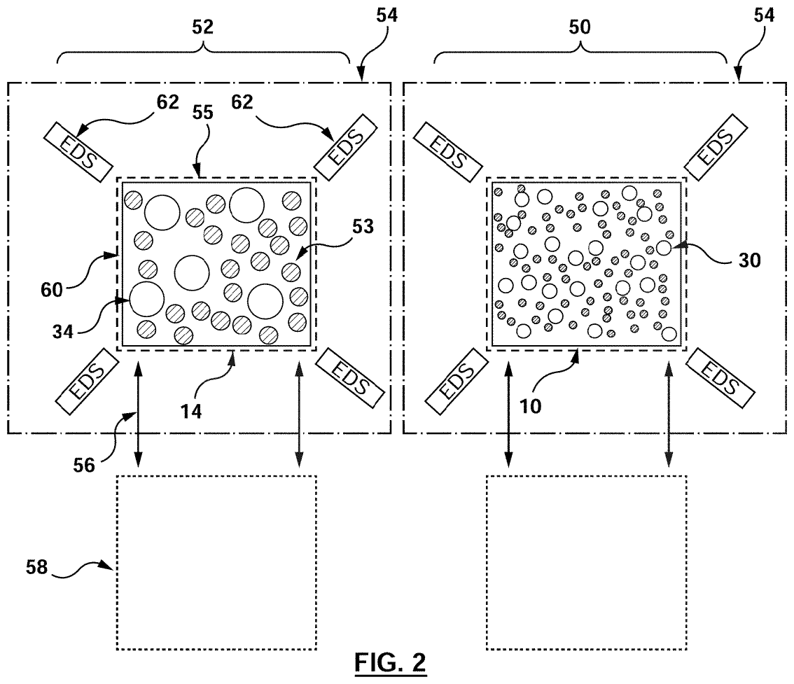

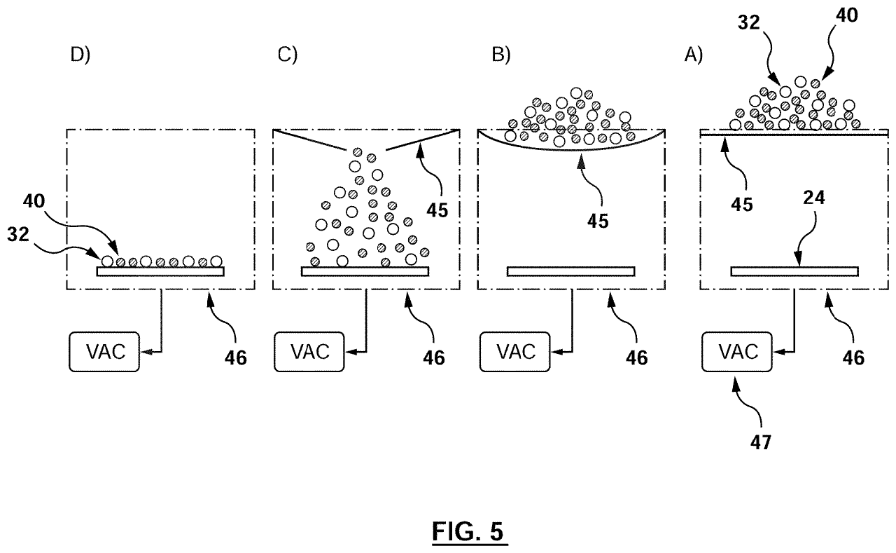

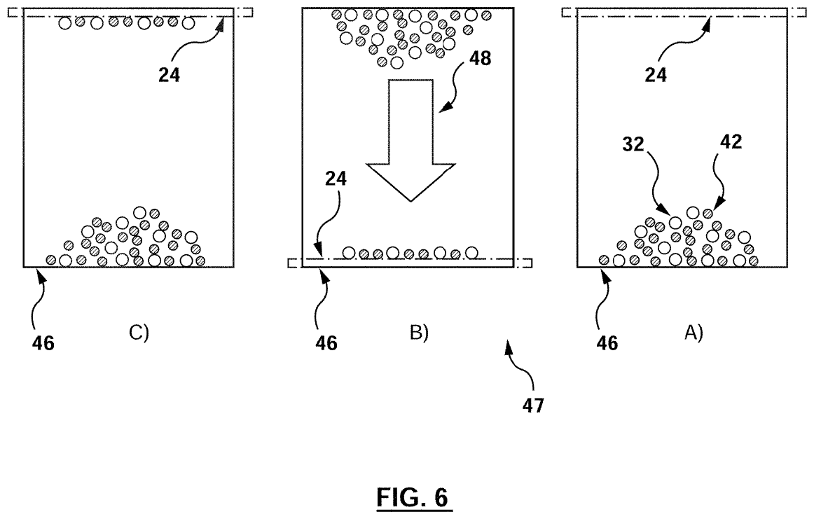

FIG. 2 schematically illustrates first and third test units 10 and 14 in plan view in respective first and third test environments from right to left at 50 and 52, with the sized sample particles in each monolayer 30, 34 having a distinct distance between one another which may be seen generally as, for illustration purposes, at least one diameter of the corresponding dispersant particle. The precise distance may vary between successive monolayers 30, 32, 34 due to a number of factors including, without limitation, the mode by which the sample particles are deposited on the substrate, which may involve, for example, vacuum assist free fall as shown in FIG. 5, and/or accelerated particle deposition as shown in FIG. 6.

Referring to FIGS. 1 and 2, the sized sample particles in each monolayer 30, 32, 34 may be statically held on the respective particle-attractant substrates 22, 24 and 26 for at least a period of time sufficient to complete an AMS analysis in the corresponding test environment, such as shown at 50, 52. For example, the particle-attractant substrate may include an adhesive layer defining a particle-attractant surface 53. Alternatively, the particle-attractant surface may be defined on, or by, a magnetic or otherwise charged or particle-attractant material substrate.

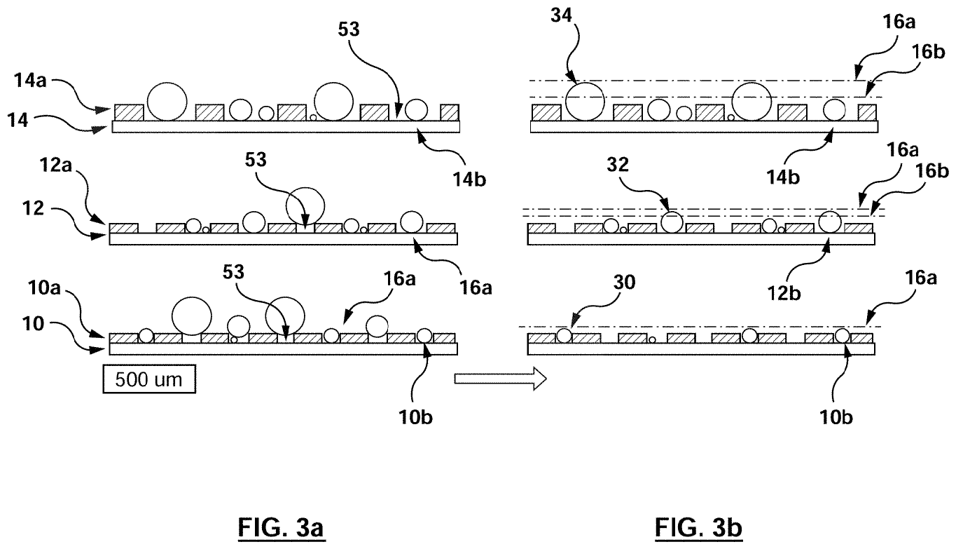

In some example embodiments, as shown in FIGS. 3a and 3b the test units 10, 12, 14, may be provided with a mask structure 10a, 12a, 14a providing respective monolayer receiving adhesive sites or sectors 10b, 12b, 14b on each particle-attractant surface 53. In this example, the mask structure 10a, 12a, 14a may be considered as providing the function of dispersing the sample particles. For each test unit 10, 12, 14, the monolayer may be defined by the particles in each sector 10, 12b, 14b. Alternatively, the substrate may be shaped to provide the sectors 10b 12b, 14b, without the need for the separate mask structure 10a, 12a, 14a. FIG. 3a shows particles following deposit on sites 10b, 12b, and 14b, while FIG. 3b shows the test units 10, 12, 14 with the loose particles removed, i.e. those shown in FIG. 3a which are not in contact with the particle-attractant surface 53. As shown in FIG. 3b, the monolayer may be defined by the particles, in each sector 10b, 12b, 14b, (or on the surface 53 of the test units 10, 12, 14 without the mask).

Further, as shown in FIG. 3b, and which may also be applicable to other example embodiments herein, the upper (or outer) extremity may be aligned with or within a depth-of-field range of a detector 62, which may be provided by way of a BES detector as a non-limiting example, defined by upper and lower (or inner) depth of field limits 16a and 16b, as shown for test units 14 and 12, wherein the lower depth of field limit may in some cases correspond with the surface 53, for example for test unit 10. In the case of test units 14 and 12, the depth of field is shown to include the upper exposed surface of the subject particles and does not include the smaller particles remaining in the sectors 12b, 14b. Thus, other smaller particles are shown to be present in several sites 10b, 12b and 14b, wherein the upper extremities of the smaller particles are not aligned with or within the depth-of-field range of the detector depth of field (or focus) range.

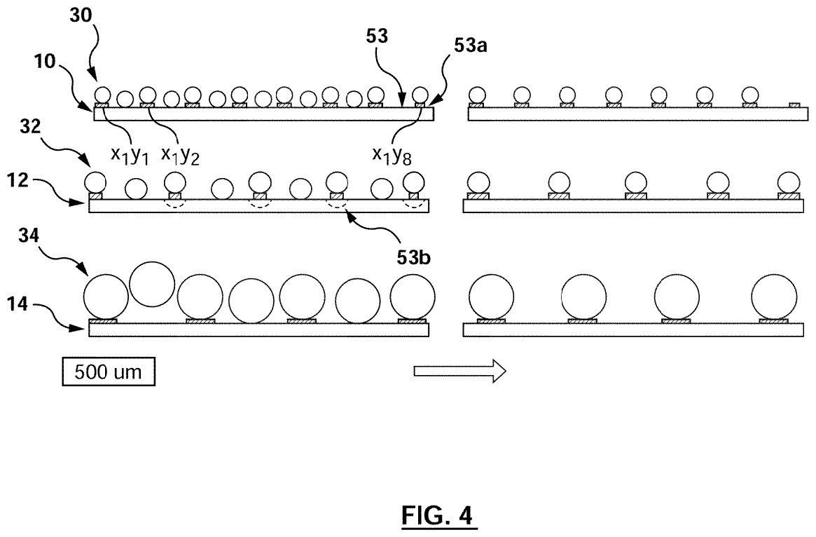

Referring to FIG. 4, in some example embodiments, the particle-attractant surface 53 may provide a designated pattern of adhesion sites 53a on the particle-attractant substrate, or sample mounting media (e.g. adhesive tape, glass, metal slide, polished surface). This may be achieved, for example, by providing a plurality of localized adhesive or magnetic sectors dots of an adhesive material forming particle-attractant substrate, or by preventing adhesion of selected parts of adhesive media, such as may be provided by adhesive tape with a mask or grid of a specific height covering the adhesive surface with openings to reveal the adhesion sites. In some instances, each site may be registrable, so as to identify a specific location of the particle (such as shown by the variables x₁y₁ to x₁,y₈ in the example of FIG. 4. In some example embodiments, in an alternative (or in some cases in addition) to the adhesion sites 53a, the particle-attractant surface may be processed to form localized open cavities, recesses or divots, as shown schematically at 53b, to seat each particle.

Referring to FIG. 2, each test environment, such as shown at 50, 52, may be calibrated and/or otherwise configured according to the characteristics of each corresponding test unit. Each test environment 50, 52 may include a test chamber 54, having a bed 55 to receive one or more of the test units 10, 12, 14, a test unit transfer structure, schematically represented by double headed arrows 56, to transfer each test unit 10, 14 between loading and testing sites 58, 60 respectively outside and inside the chamber 54, and a number of detectors 62 whose position and operating parameters may be selected and/or configured to receive radiation scattered by an exposed external profiles of the sized sample particles.

In some example embodiments, the test environments 50, 52 may be provided in the same device, which may be prepared and configured according to the test unit 10, 12, 14 under analysis. In some instances, the test environments 50, 52 may represent two operating conditions in which the same test environment is configured for two test procedures, occurring either at the same time in a single test procedure, or in subsequent test procedures. In either case, the test environment(s) may or may not be configured differently for each test procedure. In some cases, the designated settings for a particular test unit 10, 12, 14 may apply to a number of such test units. Further, the test environment may test an array of test units together. Suitable A-SEM systems may be commercially available from TESCAN Company, at https://www.tescan.com/.

### Preparation of Sample Particles

Referring to FIGS. 1 and 2, some example embodiments may provide a method of preparing a sized collection of sample particles for AMS analysis of a designated industrial value-containing material. The method may comprise depositing sized sample particles of the designated industrial material on a particle-receiving surface 53 defined on the first, second and third particle-attractant substrates 22, 24, 26 to form the respective monolayers 30, 32, 34 of the sized sample particles dispersed thereon, wherein each monolayer is configured to present the sized sample particles with outer surface profiles visible for AMS analysis.

FIG. 5 shows a technique in which, in step A, the sample particles of monolayer 32 may be mixed with dispersant particles 40 and then placed on a thin plastic foil 45 over a container 46 such as a vial, tube or other chamber forming structure. In step B, a pressure may be applied by way of vacuum source 47, to draw air out of the container 46 to cause the foil to rupture, thereby to cause (in step C), the particles to be dispersed on particle-attractant substrate 24, such as an adhesive tape located inside the chamber, under atmospheric pressure. In this case, the mixing occurs after separation of the sample particles 18 to yield the monolayer 10. This step may also be carried out on the sample particles 18 with dispersant particles of different size ranges mixed therein, prior to the separation step to yield the monolayers 30, 32 and 34.

FIG. 6 shows, at step A, an alternative technique in which the container 46, holding the mixture of sample particles making up monolayer 32, and dispersant particles 42, are to be placed on the particle-attractant substrate 24. Inverting the container at step B may then cause the particles 32 to fall onto the particle-attractant substrate 24 under gravity, as shown by the arrow 48. In step C, the container 46 may be returned to its original position with the particles 32, 42 remaining on the particle-attractant substrate 24. In some cases, a vacuum source may be applied to reduce the presence of friction that may otherwise cause differences in the travel rate of the particles toward the particle-attractant substrate 24, with the aim of having the particles 32, 42 approach the particle-attractant substrate 24 at substantially the same time, regardless of size, weight, or density.

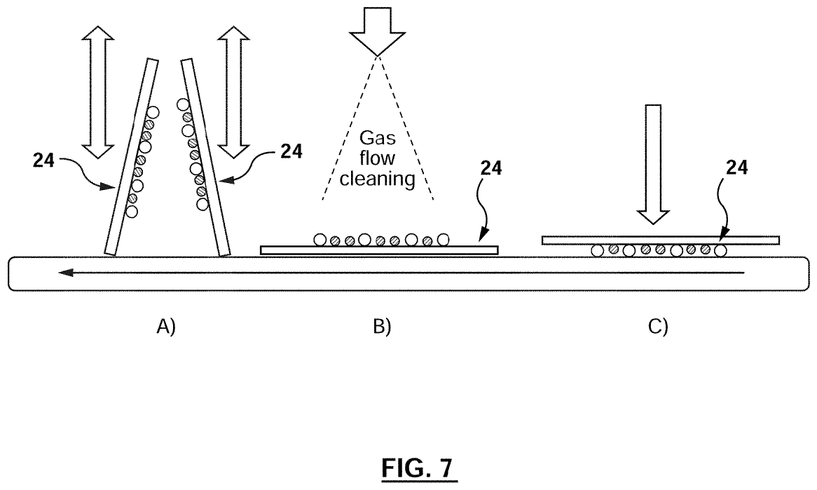

FIG. 7 represents approaches which may in some cases be deployed to establish or otherwise to improve the adhesion of particles on particle attractant surface 24, for example to improve randomness in the orientations of the particles, and/or to remove excess loose particles from the particle attractant surface 24. This may be done by applying pressure (in step C) to increase adhesion, by using a gas flow (in step B) to remove loose particles, and/or to repeatedly tap an edge of the substrate 24 against a surface at different angular orientations (as shown by the alternative positions in step A) and/or to apply a force (in step A) to improve random orientation.

In some example embodiments, appropriate sizing of sample material by physical separation of particles with wet or dry screening, cyclosizing or other means may be performed to produce designated subsets or subpopulations of particles where the maximum size may be configured to be about one to about ten times the minimum size, as expressed by the formula MAX size=MIN size*(>1 and <10), (e.g. −38 μm, 38-75 μm, 75-150 μm, 150-300 μm, 300-600 μm, 600-1200 μm, 1200-2400 μm), while minimizing the agglomerating effects of finer/smaller sample particles statically binding or bound to larger sample particles. This may be achieved, in some example embodiments, by antistatic screening and/or wet screening as discussed above.

In some example embodiments, the sized subset of particles may have a designated size distribution according to the formula:

SSPS_{MAX}=(SSPS_{MIN})A

Where
SSPS_{MAX} is the sized sample particle maximum size;
SSPS_{MIN} is the sized sample particle minimum size; and
1.0<A<10.0.

### Preparation of Test Units with Dispersed Particles

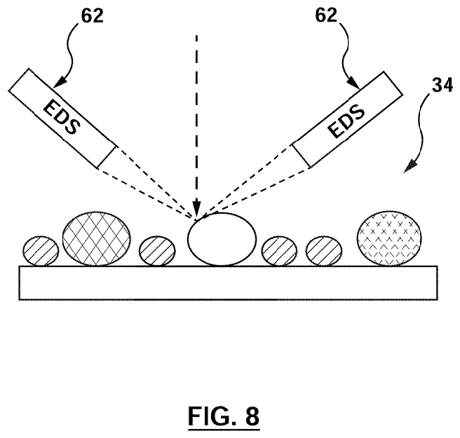

In some example embodiments, to produce a monolayer of randomly oriented and dispersed sized sample particles, the sized sample particles may first be mixed with dispersant (or filler) particles of a selected, size, shape, density and/or other characteristic. Examples of suitable materials for the dispersant particles may include graphite, hardened milled epoxy resin or other polymer, glass, ceramic, organic, or metallic materials. Transient dispersant particles, such as those which are readily soluble/removable dispersant particles (e.g. salt), or those that are volatile, or may vaporize or disintegrate (such as dry ice), may be also used in some cases to allow for its removal from the test unit after they fulfills their role to disperse the sample particles during their deposition. The ratio of the sample weight and dispersant weight may depend on the characteristics of the sized sample particles being analyzed. In some example embodiments, ratios between about 1:1 and 1:5 by weight (sample to dispersant) may be sufficient to provide operative separation of sample particles in the monolayer sample unit/sample array, so as to minimize negative effects in a later EDS analysis caused by one particle blocking the view/shading of another. Sizing of the dispersant particles may be adjusted to prevent, or otherwise minimize shading by the dispersant particles. This may be achieved, in some example embodiments, by using dispersant particles which are smaller than an average size of the sample particles, as discussed below with reference to FIG. 8. FIG. 8 shows three sample particles formed of materials of different characteristics, as represented by the different textures illustrated on each.

In some example embodiments, the dispersant particles may have a designated size distribution according to the formula:

DPS_{MAX}=(SSPS_{MAX})B

Where
DPS_{MAX} is the dispersant particle maximum size;
SSPS_{MAX} is the sized sample particle maximum size; and
0.1<B<0.9.

In some example embodiments, the sized sample particles with the dispersant particles may be placed in a test tube, mixed and transferred to the particle-attractant surface 53. This transfer may be performed by allowing the mixed particles to fall on the prepared surface or accelerate them towards the particle-attractant surface 53. In some example embodiments, the sized sample particles may be deposited on the particle-attractant substrate with the help of vacuum assisted freefall or accelerated particle deposition as discussed above. Other approaches may include the use of a spatula to disperse a small amount of material on a non-sticky surface and then pressing the tape on the surface a number of times to collect the sized sample particles on the tape.

In some example embodiments, results may be improved when excess or loose particles are removed after the fixation of the monolayer to its corresponding surface. This may be achieved in some example embodiments by delivering one or more forces either normal or tangential to the particle-attractant surface 53 to orientate the sample particles properly and/or to remove potentially remaining loose particles from the monolayer. Applying gas pressure or controlled suction to remove the loose particles and only preserve the well attached/adhered particle monolayer may also be used. Washing of the loose sample particles or dispersant particles away with suitable liquid may be also used if necessary.

### Automated Analysis

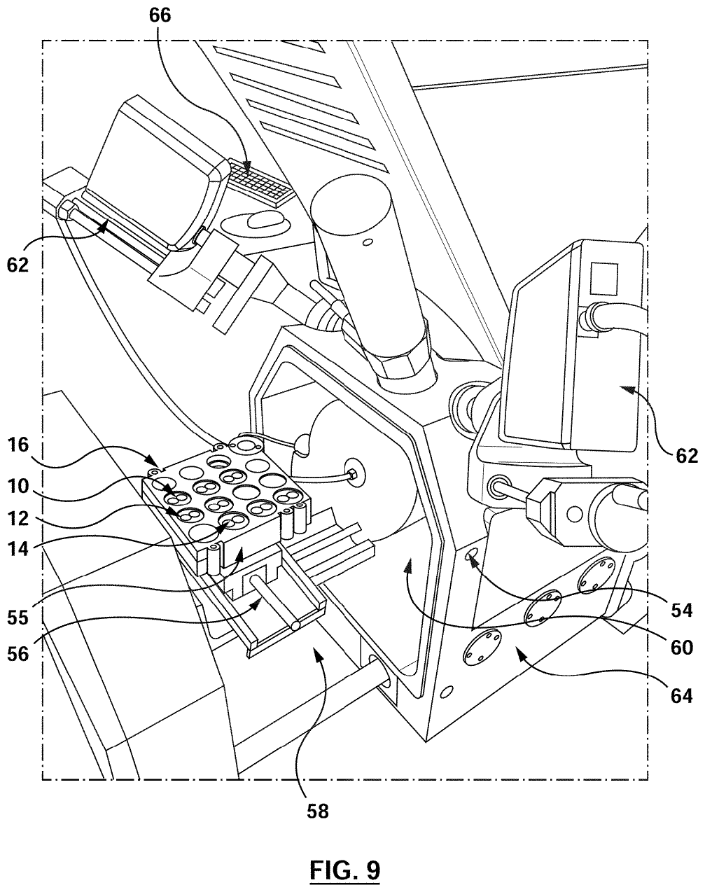

Referring to FIGS. 2 and 9, test units 10, 12, 14 may be presented to an automated SEM system with specified measurement parameters. Depending on the characteristics of the sample particles, the measurement parameters may be defined to optimize the resolution and speed at which the data are acquired, and which may yield useful modal data, (that is the proportion of individual phases in the samples), composition data and/or textural data, (that is data indicating the complexity of a particle, for instance if the particle is formed of a single mineral, or composed of multiple intergrown grains of different minerals), during the same test procedure. Multiple EDS detectors 62 may be particularly useful in this regard, as they may reduce or avoid shading and provide more relevant quantitative results from the EDS analysis. Multiple EDS detectors 62 may provide an improved collection of the scattered signal from the exposed surfaces of the particles. The collection of the scattered signal may be further improved with the selection of size ranges of each monolayer, as well as the number of test units for a particular sample. Alternative analytical techniques may be used for the (HFDMS) samples as well (e.g. LA-ICP, XRF, LIBS, microCT). As the particles are not permanently fixed in resin, after AMS measurement, the particles may be separated from the substrate and deployed in other tests, such as bulk analytical techniques like wet assay.

Some example embodiments may deploy alternative analytical techniques, such as Laser Induced Breakdown Spectroscopy (LIBS), Raman spectroscopy or other techniques such as X-Ray Fluorescence (XRF).

FIG. 8 shows a schematic side view of an example embodiment of a test environment, with two detectors 62, acting on a monolayer of sized sample particles according to third monolayer 34 in FIG. 1.

FIG. 9 illustrates an A-SEM system illustrated at 64, providing a test unit transfer unit 56 feeding a bed 55 between a loading site 58 and a testing site 60 within test chamber 54. The bed 55 is shown having, in this example, sixteen test units, of which three are identified as test units 10, 12 and 14 respectively for illustration purposes. Two detectors are shown at 62. In this case, the system 64 may communicate with one or more controllers, in this case a computer 66 for both control of the functions of the SEM system 64 and the collection of data from the EDS detectors 62 (or other suitable detectors).

While example embodiments herein provide monolayers of sized sample particles which are dispersed on the particle-attractant surface, other example embodiments may arise in which one or more of the particles on the particle-attractant surface may not necessarily be dispersed from one or more other particle, but nonetheless provide respective exposed surfaces for the AMS and other protocols described herein, and others in which example embodiments herein may be applicable. Further, some example embodiments herein may also include occurrences of sized sample particles on the particle-attractant surface which may be stacked upon one or more other such particles, without necessarily adversely affecting the results provided elsewhere by the monolayer thereof on the particle-attractant surface.

Thus, some example embodiments may yield, without limitation, one or more of the following:
a. A TAT (turn-around) time for representative sample creation may be significantly reduced, since the sample may be produced in minutes compared to hours with conventional crosscut samples, which may often be required using conventional techniques.
b. Combinations of closely sized fractions, dispersant particle usage and specific deposition are repeatable, while providing statistically representative monolayers and results.
c. Monolayer (or single layer) randomly orientated particle test units may be prepared to provide 2.5D data (i.e. providing a 2.5D environment), from the phases present in the sample particles. Textural and size data with significantly reduced stereological bias may be created when mapping the samples in A-SEM or a similar technique. A 2.5D environment may provide a three dimensional view, such as of the profile of a sample particle which is visible for AMS or other AM analysis, for example which may encompass the surface region of the sample particle that is oriented so as to be exposed to a visible field of propagation of incident electron beam radiation directly or indirectly from an electron beam emitter for the AMS analysis, so as to reflect (or generate or scatter) a signal collectable by one or more detectors. This may not, in some cases, include the outer surface profile of the sample particle that is in the shadow of the sample particle itself and which thus may not receive incident electron radiation from an electron beam. The extent of the profile of the sample may depend on the number of electron beam sources or detector configurations deployed in the AMS analysis.
d. The use of a monolayer may also avert errors caused by the potential of particle settling that can arise from differences in density and particle sizes when forming conventional crosscut samples.
e. Properties related to an actual particle surface may be better studied in relation to the process simulation of their physical behaviour.
f. Unaltered (as received) samples may be prepared and presented for different types of analysis, without the need for the additional grinding or polishing steps often required in conventional techniques to prepare crosscut samples.

### Sample Preparation and Test

### Example 1

Test units according to the present disclosure were prepared from an industrial value-containing material, in this case from an orebody from a Cu-porphyry deposit in Chile, using methods as disclosed herein. The test units were prepared using an example embodiment of the present disclosure.

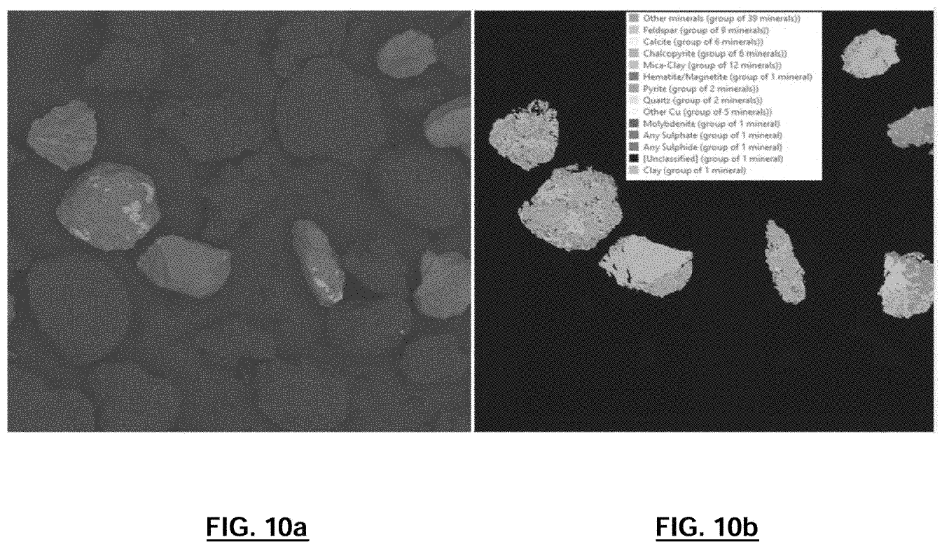

The test units were then processed using the A-SEM system 64, yielding the images of FIGS. 10a and 10b. FIGS. 10a and 10b illustrate randomly distributed dispersed deposited particles in images from BSE and after EDS based phase identification, by way of the A-SEM system 64. In this case, the A-SEM may scan the sample with a first relatively rapid pass to obtain the BSE image of FIG. 10a (in the order of minutes) and then may acquire the EDS data (in the order of hours) in the areas of interest (for example by not scanning areas between grains and the dispersant to reduce processing time) on a pixel by pixel level, thus creating a “chemical map”, as shown in FIG. 10b. Data in FIGS. 10a and 10b further enable an analysis of the surface characteristics of the sized sample particles that may be relevant for processing, and thus provide a 2.5D environment for such analysis.

FIG. 10a demonstrates the BSE image of a single field of view while FIG. 10b illustrates the mineral identification by A-SEM technology on the same single field of HFDMS/sample array. For the purpose of analysis, hundreds or thousands of individual fields may be automatically combined to provide mineral distribution (Modal) and textural data (particle size, shape and phase relations in each particle).

As illustrated in FIGS. 10a and 10b, the sample particles remain substantially in their native form, that is their form prior to separation, in the designated industrial value-containing material.

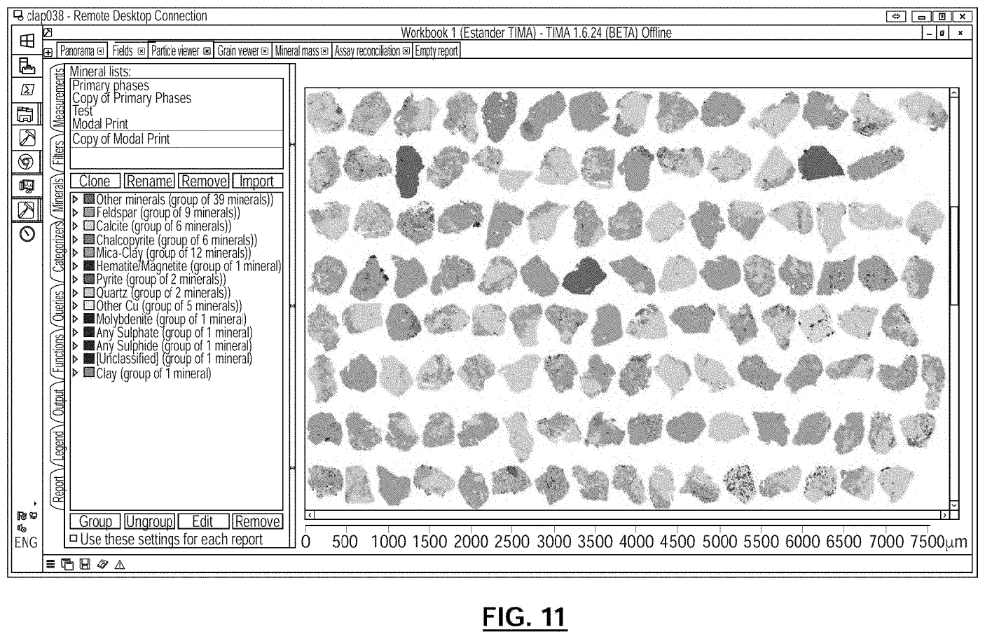

FIG. 11 represents data (particle maps with individual minerals identified by pseudo-colors) from the Cu-porphyry deposit, Chile collected with the A-SEM on the HFDMS.

### Example 2

Data produced from another subject sample material with the A-SEM system 64 yields the data shown in Table 1, which presents STD % ranging from 0.7 to 25.9. These results are comparable with the conventional 2D imaging of a crosscut sample (as shown in Example 3 below), while saving considerable time and expense as described above.

TABLE 1

| | | BLCT-hs- | BLCT-hs - | | | |
|---|---|---|---|---|---|---|
| | BLCT-hs- | 106/+75 um | 106/+75 um | | | |
| | 106/+75 um | PB2A-PB-line | PB2A-PB-line | | | |
| | PB2A-PB-line | scan2 #1 (70 | scan2 #1 (73 | | | |
| TIMA Print Replicates | scan2 #1 | exported fields) | exported fields) | AVG | STD | STD % |
| Chlorite | 3.25 | 3.44 | 3.01 | 3.2 | 0.2 | 6.7 |
| Biotite | 11.34 | 10.89 | 11.89 | 11.4 | 0.5 | 4.4 |
| Muscovite | 0.64 | 0.47 | 0.8 | 0.6 | 0.2 | 25.9 |
| Iron_oxides | 0.38 | 0.44 | 0.3 | 0.4 | 0.1 | 18.8 |
| Siderite | 0.42 | 0.47 | 0.38 | 0.4 | 0.0 | 10.7 |
| Calcite | 2.67 | 2.18 | 3.28 | 2.7 | 0.6 | 20.3 |
| Alumosilicates mix | 2.9 | 3.15 | 2.55 | 2.9 | 0.3 | 10.5 |
| Ferosaponite | 1.57 | 1.65 | 1.51 | 1.6 | 0.1 | 4.5 |
| Quartz | 12.69 | 12.99 | 12.25 | 12.6 | 0.4 | 2.9 |
| Below epoxy | 16.78 | 16.03 | 17.51 | 16.8 | 0.7 | 4.4 |
| Other silicates | 19.88 | 20.67 | 18.8 | 19.8 | 0.9 | 4.7 |
| [Unclassified] | 25.19 | 25.53 | 25.29 | 25.3 | 0.2 | 0.7 |
| The rest | 2.29 | 2.09 | 2.44 | 2.3 | 0.2 | 7.7 |
| Total | 100 | 100 | 100 | 100.0 | 0.0 | 0.0 |

### Standard Crosscut Sample Preparation and Test

### Example 3

For comparison purposes, Table 2 below shows results from a standard automated mineralogy approach using conventional crosscut samples for a different sample, and which presents STD % ranging from 0.9 to 15.7.

TABLE 2

| | QEM replicates | | | | | | | |
|---|---|---|---|---|---|---|---|---|
| | Combined | A | B | C | D | | | |
| | Measurement Type | | | | | | | |
| | | PMA | PMA | PMA | PMA | | | |
| | Measurement Name | | | | | | | |
| | | PMA + 75 um | PMA + 75 um | PMA + 75 um | PMA + 75 um | AVG | STD | STD % |
| Calculated ESD Particle Size | 58.18 | 58.76 | 59.22 | 58.37 | 56.50 | 58.2 | 1.2 | 2.1 |
| Fluorite | 16.54 | 16.87 | 16.31 | 16.96 | 16.03 | 16.5 | 0.4 | 2.7 |
| Fe-Oxides | 4.29 | 4.43 | 4.63 | 3.47 | 4.61 | 4.3 | 0.6 | 12.9 |
| Rutile | 1.34 | 1.10 | 1.58 | 1.34 | 1.36 | 1.3 | 0.2 | 14.5 |
| Biotite | 15.47 | 15.12 | 14.81 | 15.65 | 16.30 | 15.5 | 0.7 | 4.2 |
| Muscovite | 11.98 | 11.63 | 12.61 | 12.22 | 11.48 | 12.0 | 0.5 | 4.4 |
| Clays | 4.59 | 4.83 | 4.91 | 4.73 | 3.93 | 4.6 | 0.5 | 9.9 |
| Chlorite | 5.37 | 5.35 | 5.21 | 5.32 | 5.58 | 5.4 | 0.2 | 2.9 |
| Quartz | 18.78 | 18.65 | 18.88 | 18.77 | 19.02 | 18.8 | 0.2 | 0.9 |
| Feldspar | 4.14 | 4.28 | 4.07 | 4.02 | 4.17 | 4.1 | 0.1 | 2.7 |
| Amphibole | 3.02 | 2.96 | 2.87 | 3.07 | 3 19 | 3.0 | 0.1 | 4.5 |
| Epidote | 1.84 | 2.10 | 1.83 | 1.77 | 1.66 | 1.8 | 0.2 | 10.3 |
| Garnet | 1.05 | 1.01 | 0.99 | 1.06 | 1.15 | 1.1 | 0.1 | 6.8 |
| Other Silicates | 1.32 | 1.37 | 1.40 | 1.18 | 1.32 | 1.3 | 0.1 | 7.4 |
| Calcite | 4.05 | 3.69 | 4.36 | 4.08 | 4.08 | 4.1 | 0.3 | 6.8 |
| Ankerite | 1.12 | 1.22 | 0.91 | 1.30 | 1.05 | 1.1 | 0.2 | 15.7 |

Notwithstanding that the originating value-containing materials used to prepare the test units of Examples 1 and 2, and the crosscut samples of Example 3 are each of different sources, the results serve to demonstrate comparable outcomes between methods of Examples 1 and 2, according to the present disclosure, and the results of the conventional approach of Example 3. Table 1 illustrates its STD % (relative standard deviation in %), while Table 2 demonstrates the same order of STD % for different sample by the method using conventional crosscut samples, with Table 1 showing comparable results.

In some example embodiments, test units may be preserved for potential further examination by complementary techniques. In some example embodiments, this may be provided by treating a test unit with a coating, such as a thin carbon layer to be deposited onto the test unit to create an ultrathin layer of graphite atoms (such as in the order of 2-20 nm thick) to act as a conductive layer to drive excess electrons away to provide better image.

While exemplary embodiments herein refer to automated mineralogical spectral analysis, other exemplary embodiments may provide one or more of the features or elements described in the present disclosure for non-automated versions of mineralogical spectral analysis.

### Clauses

Non-limiting examples are described in the following clauses, in which the preambles “test unit” and “method” may be used interchangeably:

1. A test unit for automated mineralogical spectral analysis of a designated industrial value-containing material, the test unit comprising a particle-attractant substrate with a monolayer thereon, the monolayer comprising sized sample particles of the designated industrial value-containing material, wherein the monolayer is configured to present the sized sample particles with an outer surface profile which is visible for the spectral analysis

2. A test unit as defined in any of the preceding or following clauses, wherein the sized sample particles are dispersed on the particle-attractant substrate.

3. A test sample as defined in any of the preceding or following clauses, wherein the monolayer includes dispersant particles interspersed with the sized sample particles.

4. A test unit as defined in any of the preceding or following clauses, wherein the sized sample particles form a sized subset of sample particles from a collection of sample particles of the designated industrial value-containing material.

5. A test unit as defined in any of the preceding or following clauses, wherein the sized subset of sample particles has a designated size distribution based on a designated size ranging from about 1 to about 2400 μm in maximum diameter.

6. A test unit as defined in any of the preceding or following clauses, wherein the sized subset of particles has a designated size ranging from any one of:

from about 1 μm to about 38 μm;

from about 38 μm to about 75 μm;

from about 75 μm to about 150 μm;

from about 150 μm to about 300 μm;

from about 300 μm to about 600 μm;

from about 600 μm to about 1200 μm, and from about 1200 μm to 2400 μm.

7. A test unit as defined in any of the preceding or following clauses, wherein the sized subset of particles has a designated size distribution according to the formula:

SSPS_{MAX}=(SSPS_{MIN})A

Where

SSPS_{MAX} is the sized sample particle maximum size;

SSPS_{MIN} is the sized sample particle minimum size; and

1.0<A<10.0.

8. A test unit as defined in any of the preceding or following clauses, wherein the dispersant particles have a designated size ranging from about 10 percent to about 90 percent of a designated size of the sized sample particles.

9. A test unit as defined in any of the preceding or following clauses, wherein the dispersant particles have a designated size distribution of about 30 percent to about 90 percent of the corresponding sized sample particles.

10. A test unit as defined in any of the preceding or following clauses, wherein the dispersant particles have a designated size distribution according to the formula:

DPS_{MAX}=(SSPS_{MAX})B

Where
DPS_{MAX} is the dispersant particle maximum size;
SSPS_{MAX} is the sized sample particle maximum size; and
0.1<B<0.9.

11. A test unit as defined in any of the preceding or following clauses, wherein the dispersant particles are selected from one or more of graphite, salt, epoxy resin, glass, ceramic, metallic, organic, inorganic and/or polymeric materials.

12. A test unit as defined in any of the preceding or following clauses, wherein the sized sample particles originate from one or more steps of physical separation based on a dimensional value of the sample particles.

13. A test unit as defined in any of the preceding or following clauses, wherein the one or more steps of physical separation include one or more of wet screening, dry screening and/or cyclosizing.

14. A test unit as defined in any of the preceding or following clauses, wherein the sized sample particles are fixed to the particle-attractant substrate.

15. A test unit as defined in any of the preceding or following clauses, wherein the particle-attractant substrate includes an adhesive layer, with the sized sample particles adhered thereto.

16. A test unit as defined in any of the preceding or following clauses, wherein the particle-attractant substrate includes a magnetically or statically charged surface, with the sized sample particles adhered thereto.

17. A test unit as defined in any of the preceding or following clauses, wherein the sized sample particles and dispersant particles are fixed to the particle-attractant substrate.

18. A test unit as defined in any of the preceding or following clauses, wherein the particle-attractant substrate includes an adhesive layer, with the sized sample particles and dispersant particles adhered thereto.

19. A test unit as defined in any of the preceding or following clauses, wherein the particle-attractant substrate includes a magnetically or statically charged surface, with the sized sample particles and dispersant particles adhered thereto.

20. A test unit as defined in any of the preceding or following clauses, wherein the particle-attractant substrate includes a plurality of dispersed sites, each to receive a corresponding sized sample particle.

21. A test unit as defined in any of the preceding or following clauses, wherein the dispersed sites are defined by localized particle receiving sectors.

22. A test unit as defined in any of the preceding or following clauses, wherein the particle receiving sectors are provided by a plurality of adhesive dots.

23. A test unit as defined in any of the preceding or following clauses, wherein the particle receiving sectors are provided by a mesh structure applied to the substrate.

24. A method of preparing a test unit for mineralogical spectral analysis of a designated industrial value-containing material, comprising depositing sized sample particles of the designated industrial value-containing material on a particle-receiving surface defined on a particle-attractant substrate to form a monolayer thereon, the monolayer comprising the sized ample particles, wherein the monolayer is configured to present the sized sample particles with respective outer surface profiles which are visible for the spectral analysis.

25. A method as defined in any of the preceding or following clauses, wherein the depositing includes dispersing the sized sample particles on the particle-attractant surface.

26. A method as defined in any of the preceding or following clauses, further comprising interspersing dispersant particles with the sized sample particles in the monolayer.

27. A method as defined in any of the preceding or following clauses, further comprising forming the sized sample particles as a sized subset of sample particles from a collection of sample particles of the designated industrial value-containing material.

28. A method as defined in any of the preceding or following clauses, wherein the interspersing includes pre-mixing the dispersant particles with the sized sample particles.

29. A method as defined in any of the preceding or following clauses, wherein the sized subset of sample particles has a size distribution based on a designated size ranging from about 1 to about 2400 μm in maximum diameter.

30. A method as defined in any of the preceding or following clauses, wherein the sized subset of particles has a designated size ranging from any one of:

from about 1 μm to about 38 μm;

from about 38 μm to about 75 μm;

from about 75 μm to about 150 μm;

from about 150 μm to about 300 μm;

from about 300 μm to about 600 μm;

from about 600 μm to about 1200 μm, and

from about 1200 μm to 2400 μm.

31. A method as defined in any of the preceding or following clauses, wherein the sized subset of particles has a designated size distribution according to the formula:

SSPS_{MAX}=(SSPS_{MIN})A

Where

SSPS_{MAX} is the sized sample particle maximum size;

SSPS_{MIN} is the sized sample particle minimum size; and

1.0<A<10.0.

32. A method as defined in any of the preceding or following clauses, wherein the dispersant particles have a designated size ranging from about 10 percent to about 90 percent of the designated size of the sized sample particles.

33. A method as defined in any of the preceding or following clauses, wherein the sized sample particles and/or dispersant particles have a designated size distribution of about 30 percent to about 90 percent.

34. A method as defined in any of the preceding or following clauses, wherein the dispersant particles have a designated size distribution according to the formula:

DPS_{MAX}=(SSPS_{MAX})B

Where

DPS_{MAX} is the dispersant particle maximum size;

SSPS_{MAX} is the sized sample particle maximum size; and

0.1<B<0.9.

35. A method as defined in any of the preceding or following clauses, wherein the dispersant particles are selected from one or more of graphite, hardened milled epoxy resin, hardened glass, ceramic, metallic, organic, inorganic and/or polymeric materials.

36. A method as defined in any of the preceding or following clauses, further comprising fixing the sized sample particles to the particle-attractant substrate.

37. A method as defined in any of the preceding or following clauses, wherein the particle-attractant substrate includes an adhesive layer, wherein the fixing includes adhering the sized sample particles thereto.

38. A method as defined in any of the preceding or following clauses, further comprising fixing the sized sample particles and dispersant particles to the particle-attractant substrate.

39. A method as defined in any of the preceding or following clauses, wherein the particle-attractant substrate includes an adhesive layer, wherein the fixing includes adhering the sized sample particles and dispersant particles thereto.

40. A method as defined in any of the preceding or following clauses, further comprising wet screening, dry screening and/or cyclosizing the material to obtain the sized sample particles.

41. A method as defined in any of the preceding or following clauses, wherein the interspersing includes depositing dispersant particles with the sample particles on the particle-receiving surface.

42. A method as defined in any of the preceding or following clauses, wherein prior to the depositing of the dispersant particles, further comprising mixing the sample particles with the dispersant particles.

43. A method as defined in any of the preceding or following clauses, further comprising forming a plurality of dispersed sites on the particle-attractant substrate, for each to receive a corresponding sized sample particle.

44. A method as defined in any of the preceding or following clauses, wherein the forming includes forming localized particle receiving sectors.

45. A method as defined in any of the preceding or following clauses, wherein the forming includes forming a plurality of adhesive dots.

46. A method as defined in any of the preceding or following clauses, wherein the forming includes applying a mesh structure to the substrate.

47. A test unit for mineralogical spectral analysis of a designated industrial value-containing material, the test unit comprising a particle-attractant substrate with a monolayer thereon, the monolayer comprising sized sample particles of the designated industrial value-containing material dispersed, wherein at least some of the sample particles in the monolayer are dispersed and the monolayer is configured to present the sized sample particles with an outer surface profile which is visible, within a depth of field range of one or more detectors, or otherwise suitable for the spectral analysis.

48. A test unit for mineralogical spectral analysis of a designated industrial value-containing material, comprising a particle-attractant substrate with a monolayer, the monolayer comprising sized sample particles of the designated industrial value-containing material thereon, wherein the monolayer is configured to present the sized sample particles with respective outer surface profiles which are visible, within a designated depth of field range of one or more spectral analysis detectors.

49. A test unit as defined in any of the preceding or following clauses, wherein the depth of field range includes an outer limit beyond an outer extremity of the particles, and an inner limit intermediate the outer extremity and an inner extremity.

50. A test unit as defined in any of the preceding or following clauses, wherein the inner extremity corresponds to a reference plane or surface of the particle-attractant substrate.

51. A method for analyzing an industrial value-containing material, comprising initiating spectral analysis on a test unit as defined in any of the preceding or following clauses.

52. A method for analyzing an industrial value-containing material, comprising preparing a test array of one or more test units as defined in any of the preceding or following clauses, wherein each of the one or more test units in the test array includes a plurality of corresponding sized sample particles of a designated size range, initiating spectral analysis on the test array to collect data on each of the at least one test unit, and determining value-containing material content and/or properties therefrom.

53. A method as defined in any of the preceding or following clauses, further comprising assembling datasets for each test unit, so as to create a number of datasets for the test array, and following the initiating, forming a cumulative dataset for the test array from the number of datasets.

54. A method as defined in any of the preceding or following clauses, further comprising configuring one or more spectral analysis detectors with a designated depth of field range to collect data from a visible outer surface of the sized particles.

55. A method as defined in any of the preceding or following clauses, wherein the configuring incudes setting a depth of field outer limit to be beyond an outer extremity of the particles, and a depth of field inner limit intermediate the outer extremity and an inner extremity.

56. A method as defined in any of the preceding or following clauses, wherein the configuring includes setting the depth of field inner limit to correspond to a reference plane or of the particle-receiving substrate.

57. A test unit for automated mineralogical spectral analysis of a designated industrial value-containing material, comprising a particle-attractant substrate with a monolayer of sized sample particles of the designated industrial value-containing material thereon, wherein the monolayer is configured to present the sized sample particles with an outer surface profile which is visible for the spectral analysis.

58. A test unit for automated mineralogical spectral analysis of a designated industrial value-containing material, comprising a particle-attractant substrate with a monolayer segment of sized sample particles of the designated industrial value-containing material thereon, wherein the monolayer segment is configured to present the sized sample particles with an outer surface profile which is visible for the spectral analysis.

59. A test unit as defined in any of the preceding or following clauses, wherein the least one monolayer segment includes a first monolayer segment of sized sample particles of a first size range and a second monolayer segment of sized sample particles of a second size range.

60. A test unit as defined in any of the preceding or following clauses, further comprising a plurality of dispersant particles interspersed with the sized sample particles in the monolayer.

61. A test unit as defined in any of the preceding or following clauses, wherein the dispersant particles are transient and selected from any one or more of particles that are soluble, volatile, removable, or configured to vaporize or disintegrate.

62. A test unit as defined in any of the preceding or following clauses, wherein the dispersant particles are formed from salt or dry ice.

63. A test unit defined in any of the preceding or following clauses, wherein the designated industrial value-containing material comprises one or more of:
non-metallic ores including limestone, manganese, mica, gypsum, coal, dolomite, phosphate, salt, granite;
metal ores including iron, nickel, lead, zinc copper, aluminium, tin, tungsten, molybdenum, tantalum, cobalt, bismuth, cadmium, titanium, zirconium, antimony, manganese, magnesium, lithium, uranium, thorium, beryllium, chromium, germanium, vanadium, gallium, hafnium, indium, niobium, rhenium, and thallium;
precious metal ores including gold, silver, palladium, and the platinum group metals including ruthenium, rhodium, palladium, osmium, iridium, and platinum,
precious stones including diamond, ruby and/or sapphire;
host materials of one or more metal, precious metals, precious stones, in one or more feed, concentrate and/or tailings forms; and
processing, smelting, rafting products/materials thereof including, without limitation, matte, slag and/or slimes.

64. A method of preparing a test unit for mineralogical spectral analysis of a designated industrial value-containing material, comprising depositing sized sample particles of the designated industrial value-containing material on a particle-receiving surface defined on a particle-attractant substrate to form a monolayer of the sized sample particles thereon, wherein the monolayer is configured to present the sized sample particles with respective outer surface profiles which are visible for the spectral analysis.

While the present disclosure describes various example embodiments, the disclosure is not so limited. To the contrary, the disclosure is intended to cover various modifications and equivalent arrangements, as will be readily appreciated by the person of ordinary skill in the art.

1. A test unit for mineralogical spectral analysis of a designated industrial value-containing material, comprising a particle-attractant substrate with a monolayer, the monolayer comprising sized sample particles of the designated industrial value-containing material thereon, wherein the monolayer is configured to present the sized sample particles with respective outer surface profiles which are visible, within a designated depth of field range of one or more spectral analysis detectors.

2. A test unit as defined in claim 1, wherein the depth of field range includes an outer limit beyond an outer extremity of the particles, and an inner limit intermediate the outer extremity and an inner extremity.

3. A test unit as defined in claim 2, wherein the inner extremity corresponds to a reference plane or surface of the particle-attractant substrate.

4. A test unit as defined in claim 1, wherein the monolayer includes dispersant particles interspersed with the sized sample particles.

5. A test unit as defined in claim 1, wherein the sized sample particles form a sized subset of sample particles from a collection of sample particles of the designated industrial value-containing material.

6. A test unit as defined in claim 5, wherein the sized subset of sample particles has a designated size distribution based on a designated size ranging from about 1 to about 2400 μm in maximum diameter.

7. A test unit as defined in claim 5, wherein the sized subset of particles has a designated size ranging from any one of:
from about 1 μm to about 38 μm;
from about 38 μm to about 75 μm;
from about 75 μm to about 150 μm;
from about 150 μm to about 300 μm;
from about 300 μm to about 600 μm;
from about 600 μm to about 1200 μm, and
from about 1200 μm to 2400 μm.

8. A test unit as defined in claim 5, wherein the sized subset of particles has a designated size distribution according to the formula:
SSPS_{MAX}=(SSPS_{MIN})A
Where
SSPS_{MAX} is the sized sample particle maximum size;
SSPS_{MIN} is the sized sample particle minimum size; and
1.0<A<10.0.

9. A test unit as defined in claim 4, wherein the dispersant particles have a designated size ranging from about 10 percent to about 90 percent of a designated size of the sized sample particles.

10. A test unit as defined in claim 4, wherein the dispersant particles have a designated size distribution of about 30 percent to about 90 percent of the corresponding sized sample particles.

11. A test unit as defined in claim 4, wherein the dispersant particles have a designated size distribution according to the formula:
DPS_{MAX}=(SSPS_{MAX})B
Where
DPS_{MAX} is the dispersant particle maximum size;
SSPS_{MAX} is the sized sample particle maximum size; and
0.1<B<0.9.

12. A test unit as defined in claim 4, wherein the dispersant particles are selected from one or more of graphite, salt, epoxy resin, glass, ceramic, metallic, organic, inorganic and/or polymeric materials.

13. A test unit as defined in claim 1, wherein the sized sample particles originate from one or more steps of physical separation based on a dimensional value of the sample particles.

14. A test unit as defined in claim 13, wherein the one or more steps of physical separation include one or more of wet screening, dry screening and/or cyclosizing.

15. A test unit as defined in claim 14, wherein the sized sample particles are fixed to the particle-attractant substrate.

16. A test unit as defined in claim 15, wherein the particle-attractant substrate includes an adhesive layer, with the sized sample particles adhered thereto.

17. A test unit as defined in claim 15, wherein the particle-attractant substrate includes a magnetically or statically charged surface, with the sized sample particles adhered thereto.

18. A test unit as defined in claim 1, wherein the dispersed sites are defined by localized particle receiving sectors.

19. A test unit as defined in claim 18, wherein the particle receiving sectors are provided by a plurality of adhesive dots.

20. A test unit as defined in claim 18, wherein the particle receiving sectors are provided by a mesh structure applied to the substrate.

21. A test unit as defined in claim 4, wherein the dispersant particles are transient and selected from any one or more of particles that are soluble, volatile, removable, or configured to vaporize or disintegrate.

22. A test unit as defined in claim 21, wherein the dispersant particles are formed from salt or dry ice.

23. A test unit as defined in claim 2, wherein the designated industrial value-containing material comprises one or more of:
non-metallic ores including limestone, manganese, mica, gypsum, coal, dolomite, phosphate, salt, granite;
metal ores including iron, nickel, lead, zinc copper, aluminium, tin, tungsten, molybdenum, tantalum, cobalt, bismuth, cadmium, titanium, zirconium, antimony, manganese, magnesium, lithium, uranium, thorium, beryllium, chromium, germanium, vanadium, gallium, hafnium, indium, niobium, rhenium, and thallium;
precious metal ores including gold, silver, palladium, and the platinum group metals including ruthenium, rhodium, palladium, osmium, iridium, and platinum,
precious stones including diamond, ruby and/or sapphire;
host materials of one or more metal, precious metals, precious stones, in one or more feed, concentrate and/or tailings forms; and
processing, smelting, rafting products/materials thereof including, without limitation, matte, slag and/or slimes.
